(19) United States
(12) Patent Application Publication
(10) Pub. No.: US 2001/0044132 A1
(43) Pub. Date: Nov. 22, 2001

(54) Method for calculating and estimating the statistical significance of gene expression ratios

(76) Inventor: Thomas M. Houts, Gilroy, CA (US)

Correspondence Address: AMERSHAM PHARMACIA BIOTECH INC., PATENT DEPARTMENT, P O BOX 1327, 800 CENTENNIAL AVENUE, PISCATAWAY, NJ, 088551327

(21) Appl. No.: 09/850,296

(22) Filed: May 7, 2001

Related U.S. Application Data
(60) Provisional application No. 60/203,659, filed on May 12, 2000.

(51) Int. Cl. C12P 21/06; G01N 33/48

(52) U.S. Cl. 435/69.1; 702/19

(57) ABSTRACT

A method for calculating gene expression ratios that comprises the steps of spotting a target nucleic acid sequence onto a first area and a second area of a substrate, contacting under hybridization conditions the target nucleic acid contained on the first area and second area of the substrate with a sample containing a mixture of nucleic acids labeled with at least two different labels, measuring a first signal that corresponds to a first label from the first area and second signal corresponding to a second label from the first area of the substrate, determining the log ratio of the first and second signals, fitting a nonlinear relationship relating the uncorrected log ratio to the logarithmic value of a function of the first signal, subtracting the nonlinear relationship from the uncorrected log ratio to obtain a normalized log ratio, and calculating the normalized gene expression ratio as the anti-log of the normalized log ratio. The disclosed method may further comprise the step of estimating the statistical significance of normalized gene expression ratios.

### FIELD OF THE INVENTION

The instant disclosure pertains to a method for calculating and estimating the statistical significance of gene expression ratios. In particular, the instant disclosure pertains to a method for calculating and estimating the significance of gene expression ratios obtained from microarray analysis data.

### BACKGROUND OF THE INVENTION

Microarray technology is a powerful analytical technique for genetic research. Microarrays are arrays of gene fragments attached to substrates, usually glass chips. The gene fragments are typically spotted onto a chip in a grid arrangement. Hybridization reactions between the sequences on the microarray and a fluorescently labeled nucleic acid sample can be used to identify gene expression levels. After hybridization, the chips are analyzed using high-speed fluorescent detectors and the intensity of each spot is quantified. Many commercially available detectors can detect three, four, or five different colors of fluorescent labels, thus allowing for analysis of multiple samples in a single experiment. The location and intensity of each spot reveals the identity and amount of each sequence present in the sample. The total fluorescence intensity of a spot is proportional to the expression level of a gene. Because each microarray can contain thousands of gene fragments, it is possible to obtain data for an entire genome in a single experiment.

Different variables and parameters (e.g., slide quality, pin quality, amount of DNA spotted, accuracy of arraying device, dye characteristics, scanner quality, quantification software characteristics, etc.) can affect the measured expression levels. Normalization is necessary to remove or minimize the expression differences due to the variability in these parameters. Effective normalization and estimation of imprecision in microarray data analysis have been challenges facing microarray technology for some time. This has been true, even though there is an enormous amount of data (up to 4600 spots in duplicate). Moreover, data from a wide range of microarray experiments has revealed that normalization is not constant. For example, Hammond, T. G., et al., in Gene Expression in Space, 5 NATURE MEDICINE 359 (1999), show observation of non-constant normalization without suggesting any approach to resolution. A poster entitled, “Robust Normalisation of Microarray Data Over Multiple Experiments, “presented at the 1999 Nature Genetics Microarray Conference in Scottsdale, Ariz., Morrison, N. et al. suggests log transformation of signal and demonstrates log normal distribution of signals, but suggests making comparisons within color by normalizing to the distribution. The normalization, however, is only fundamentally a single channel normalization.

Additionally, imprecision (i.e., the standard deviation of the log of the ratio of the two signals for replicate genes) is not constant, but has been found to increase toward lower signal. These relationships also vary from experiment to experiment.

There is therefore a need for improvement in the area of calculating and estimating the statistical significance of gene expression ratios.

### SUMMARY OF THE INVENTION

In view of the needs of the art, the present invention provides a method of calculating gene expression ratios whereby log ratio data for two signals is related to the log of some function of at least one of those signals, an exponential curve is fitted to the data, and the data is normalized by subtracting the fitted curve from each data point.

In one embodiment, the present invention provides method for calculating gene expression ratios that comprises spotting a target nucleic acid sequence onto a first area and a second area of a substrate; contacting under hybridization conditions the target nucleic acid contained on the first area and second area of the substrate with a sample containing a mixture of nucleic acids labeled with at least two different labels; measuring a first signal that corresponds to the first label from the first area and second signal corresponding to the second label from the first area of the substrate; converting the signals of the two labels to logarithmic values; subtracting the log of the first signal from the log of the second signal to create an uncorrected log ratio; fitting a nonlinear (exponential) relationship relating uncorrected log ratio to the log of the first signal; subtracting the fitted uncorrected log ratio based on the corresponding log of the first signal from the uncorrected log ratio to obtain a normalized log ratio; and calculating the normalized gene expression ratio as the anti-log of the normalized log ratio. The first label may be a Cy5 dye and the second label may be a Cy3 dye.

The present invention further provides a method for calculating gene expression ratios according to either of the embodiments disclosed above, further comprising the step of estimating the statistical significance of normalized gene expression ratios that comprises: a) creating at least 10 bins spanning the range of the function of at least one of the first and second signals; b) calculating the square root of the average variance for each bin created in step a); and c) estimating the relationship between the function of at least one of the first and second signals and the square root of the average variance using the equation

=a+b exp(−x/c)

wherein y=square root of the average variance and x is the function of at least one of the first and second signals, and a, b, and c are regression constants; this value representing the fitted square root of the average variance which represents “s pooled” or σ.

The present invention even further pertains to a method wherein both the average variance and regression are weighted to reflect degrees of freedom.

The present invention still further provides a method for estimating the significance of normalized gene expression ratios from any method for calculating gene expression ratios that comprises: spotting at least one target in at least two or more replicates; calculating the normalized log ratio for each target; calculating the s pooled or variance as a function of signal intensity; and using the value determined in step c) in one or both of the following: the determination of confidence intervals for gene expression ratios, or flagging unusually variable sets of replicate ratios. The present invention even still further provides that the signal intensity used to calculate the relationship between spooled and signal intensity is the average signal intensity.

### BRIEF DESCRIPTION OF THE SEVERAL VIEWS OF THE DRAWINGS

FIG. 1 is a graph showing the distribution of log (Cy5 signals) in a typical two-color gene expression experiment.

FIG. 2 is a graph showing the average log ratio (=log (Cy3/Cy5)) for each of 50 bins for the same experiment as in FIG. 1, plotted as a function of the average log (Cy5 signal) for the bin. Also shown is an exponential curve fitted to the data. For bins containing more than about 50 members, the fitted curve is very close to the average log ratio for that bin. Deviations from the fitted curve for bins with fewer members is not unexpected, and might be due to differential expression of the genes represented in the bin.

FIG. 3 is a graph showing the relationship between s-pooled for normalized log ratios (sqrtvbrd1=square root of the average variance for the bin) and log (Cy5 signal) for the experiment in FIGS. 1 and 2. An exponential curve approximates the relationship, as indicated by the plus symbols. Also shown is the relationship between the s-pooled for uncorrected log ratios (sqwrtvucd1) and log (Cy5 signal).

### DETAILED DESCRIPTION OF THE PREFERRED EMBODIMENT

The instant disclosure is based on identification of an equation (or model) with the flexibility to describe the general shape of a relationship, and an approach to extracting this relationship from any microarray experiments. A surprising benefit of this approach is that precision of the gene expression ratios is better than that which is possible by averaging signals across spots, or normalizing to an average ratio from multiple spots.

Briefly, at least two spots of a target nucleic acid sequence are spotted onto a substrate. The spots are contacted under hybridization conditions with a sample containing a mixture of nucleic acids labeled with at least two different labels, such mRNA of a first tissue labeled with Cy3 (green) fluorescent dye and mRNA from a second tissue labeled with Cy5 (red) fluorescent dye. The signals from each label from each spot are measured. The signals measure the intensity of the fluorescence of the spot. A log transformation is taken of the signal from each spot; the difference in log(signal) for the two channels is the log of the ratio of the two signals. Working in log space is advantageous for two reasons: 1) the distribution of log signals is normal, as is the distribution of differences in log signals, making it easier to predict distributions; and 2) the standard deviation of the difference in log signal can be used to calculate confidence intervals for the ratios.

A binned weighted regression is used to estimate the non-linear normalization relationship. The bins being equal-sized divisions spanning the range of log(Cy5 signals). Desirably, at least 10 bins are employed and more desirably, about 50 bins are employed. Weighting is according to the number of spots in each bin. The equation form which fits over a wide range of conditions is y=a+b*exp(−x/c), where y is the mean in each bin of the uncorrected difference in log signals between Cy3 and Cy5 for all the spots in the bin, and x is the average log (Cy5 signal) for the bin, and the variables a, b, and c, are parameters of the fitted exponential curve. It is further contemplated by the present invention that normalization need not be estimated using a bin-weighted calculation.

From the normalization equation above, for each spot a normalized difference in log signals is obtained by subtracting the expected difference in log signals for that Cy5 signal from the uncorrected difference in log signals. This normalized difference is effectively the corrected log of the ratio of the two signals for the spot. The normalized or corrected gene expression ratio can be calculated as the anti-log of the normalized or corrected log ratio.

Imprecision is estimated in a similar manner. Each target is spotted and hybridized in duplicate. Hence, each target provides a Cy3 and Cy5 signal for the left spot, and the same for the right spot. We can calculate the variance of the normalized differences in log signal for that pair of duplicates. An estimate of variance from duplicates is poor, but these estimates can be pooled (averaged) within the same regression to estimate the relationship between variance and the Cy5 signal level. The equation form which has been reasonably successful is y=a+b*exp(−x/c), where y is the square root of the average variance for a bin, and x is the average log signal for a bin. Weights are once again according to the number of points in each bin.

A good normalization combined with a good estimate of the imprecision allows estimation of confidence intervals or significance scores (p-values) for individual targets. Also, tests can be developed to flag duplicates with unusually poor precision.

This concept can be extended to the use of replicates for a subset of the spots to allow estimation of these relationships, and then use these relationships to normalize and estimate confidence intervals for genes which are only present without replication.

The present invention also contemplates that the signal data from the dye labels may alternatively be related and normalized to other functions of the signals produced from one or both of the dyes. For example, the signal data from the two dye labels may be related and normalized to only the signal of the second label, or the Cy3 dye. Alternatively still, the signal data from the two dye labels may be related and normalized to a mathematical expression of the two signals, such as the average between the two signals, their maximum signal, their minimum signal. Other expressions may be employed including, by way of illustration and not of limitation, the square root of the product of the two signals. This last expression has been found useful for fitting imprecision as a function of signal as it weights the lower signals.

Further extension of this concept could include use of estimates of regression uncertainty for both the correction factor and the imprecision to further improve reliability of the confidence intervals. The following examples are for illustration purposes only and should not be used in any way to limit the appended claims.

### EXAMPLES

The present invention contemplates that data obtained from gene expression experiments may be obtained by conventional means for the art and stored electronically as is known in the art. Raw data may be stored, for example, in spreadsheet files and manipulated as required or taught by the present invention. The present invention may be applied when analyzing data from several slide types. Precision is generally better employing the present normalization method than without normalization. As confidence intervals for the expression ratios vary substantially from slide to slide as well as by signal intensity; the present methods allow correct estimation of the confidence intervals in the gene expression ratios based on the data contained within each experiment.

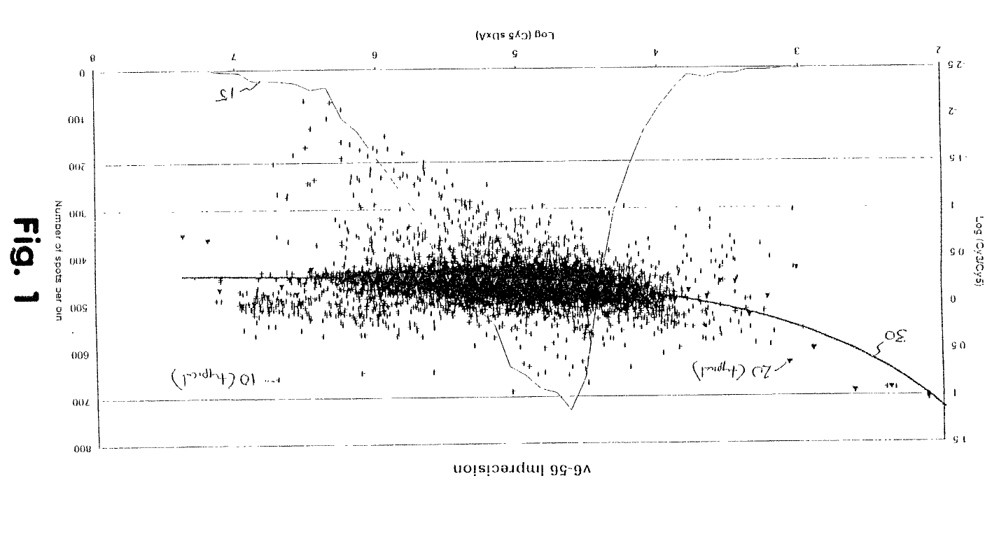

FIG. 1 represents the results from a two-color gene expression experiment. There are 4192 targets spotted in duplicate, which are hybridized to a mixture of Cy3 labeled cDNA prepared from the mRNA of one tissue, and Cy5 labeled cDNA prepared from the mRNA of a different tissue. The signal intensity for each of the two fluorescent dyes associated with each target was measured and quantified.

FIG. 1 depicts a graph which plots the log of the ratio (Cy3 signal)/(Cy5 signal) against the log of the Cy5 signal for each spot. The “plus” signs 10 represent each individual nonzero spot (9183). Based on log Cy5 signal, the data were grouped into 50 bins. The thin line 15 represents the number of spots in each bin. The triangles 20 are the average log ratio for each bin, plotted against the average log(Cy5 signal) for that bin. It is easy to see that the average log ratios do not fit a straight, horizontal line representing constant normalization. The deviation in this case, from zero at a logsignal of 3.9 to about −0.3 at the higher logsignals, represents a deviation in the normalization target of about a factor of two over the bulk of the data. The thicker line 30 represents an exponential decay fit to all 9183 non-zero spots. Line 30 matches the average log ratios for the bins very well, provided the number of genes on which the average is based exceeds, in the example of FIG. 1, 50. The present invention contemplates that the number genes on which the average is based may be selected on a case by case basis.

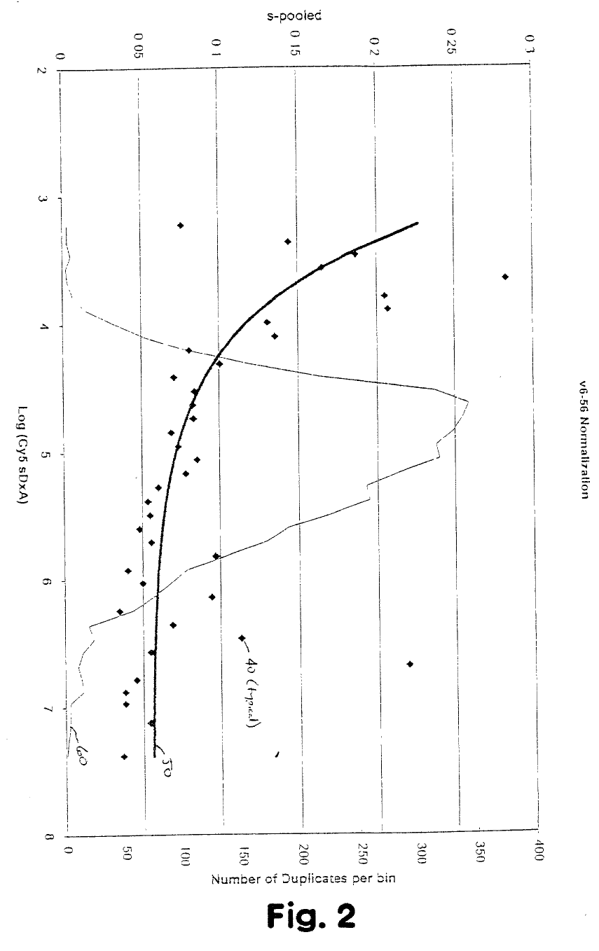

FIG. 2 represents the imprecision in the normalized gene expression ratios calculated from the same experiment as in FIG. 1. Using the same bins of log(Cy5 signal) as used in FIG. 1, s-pooled for the bin was determined by calculating the square root of the average variance of duplicate log ratios within the bin. The graph shows the s-pooled for the bin plotted against the average log (Cy5 signal) for the bin as individual diamonds 40, and also shows the fitted exponential curve 50. Line 60 depicts the number of duplicates in each bin, which is also the degrees of freedom for the s-pooled calculated in that bin. It can be seen that imprecision becomes greater at lower signal levels.

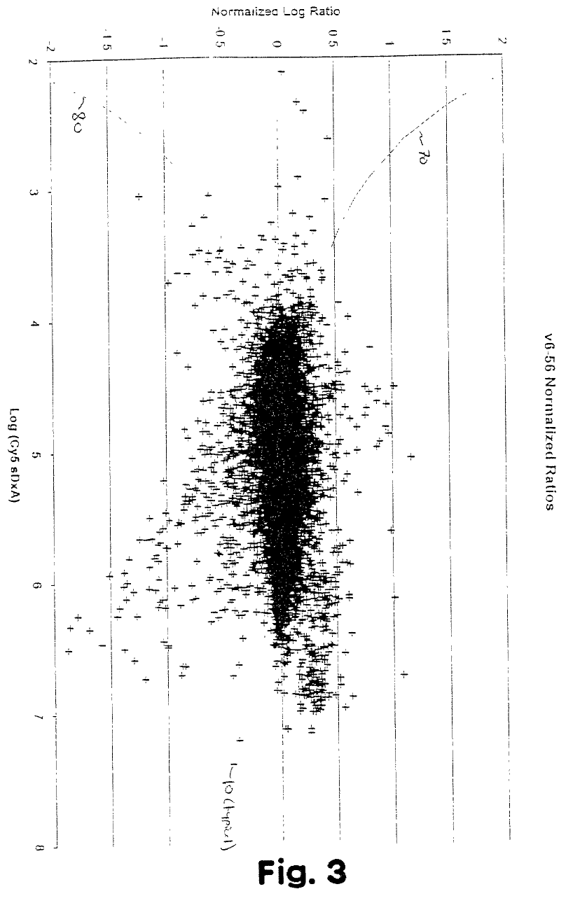

FIG. 3 plots the normalized log ratios for each individual spot 10 in the experiment described in FIG. 1. Lines 70 and 80 depict the 99% confidence limits for a single replicate based on the calculated relationship between s-pooled and log (Cy5 signal). There is an improvement in precision of the log ratios after normalization, particularly at the lower signal values where the width of the confidence interval is greater.

Although a number of embodiments are described in detail by the above examples, the instant invention is not limited to such specific examples. Various modifications will be readily apparent to one of ordinary skill in the art and fall within the spirit and scope of the appended claims.

What is claimed is:

1. A method for calculating gene expression ratios that comprises:
a) spotting a target nucleic acid sequence onto a first area and a second area of a substrate;
b) contacting under hybridization conditions said target nucleic acid contained on the first area and second area of said substrate with a sample containing a mixture of nucleic acids labeled with at least two different labels;
c) measuring a first signal that corresponds to a first label from said first area and second signal corresponding to a second label from said first area of said substrate;
d) determining the log ratio of said first signal to said second signal;
e) fitting a nonlinear relationship relating uncorrected log ratio to the logarithmic value of a function of said first signal;
f) subtracting said nonlinear relationship from the uncorrected log ratio to obtain a normalized log ratio; and
g) calculating the normalized gene expression ratio as the anti-log of the normalized log ratio.

2. The method of claim 1, wherein said fitting step further comprises fitting a nonlinear relationship relating uncorrected logarithm of the ratio of said first signal to said second signal to the logarithmic value of said first signal.

3. The method of claim 1, wherein said fitting step further comprises fitting a nonlinear relationship relating uncorrected logarithm of the ratio of said first signal to said second signal to the logarithmic value of the sum of said first and second signals.

4. The method of claim 1, wherein said fitting step further comprises fitting a nonlinear relationship relating uncorrected logarithm of the ratio of said first signal to said second signal to the logarithmic value of the average of said first and second signals.

5. The method of claim 1, wherein said fitting step further comprises fitting a nonlinear relationship relating uncorrected logarithm of the ratio of said first signal to said second signal to the logarithmic value of the maximum of said first and second signals.

6. The method of claim 1, wherein said fitting step further comprises fitting a nonlinear relationship relating uncorrected logarithm of the ratio of said first signal to said second signal to the logarithmic value of the minimum of said first and second signals.

7. The method of claim 1, wherein said fitting step further comprises fitting a nonlinear relationship relating uncorrected logarithm of the ratio of said first signal to said second signal to the logarithmic value of the square root of the product of said first and second signals.

8. The method of claim 1, wherein said fitting step further comprises fitting an exponential relationship relating the uncorrected logarithm of the ratio of said first signal to said second signal to the logarithmic value of said first signal.

9. The method of claim 1, wherein said determining step further comprises the steps of:
a) converting said first and second signals to logarithmic values; and
b) subtracting the logarithmic value of said first signal from the logarithmic value of said second signal to create an uncorrected log ratio.

10. The method of claim 1, wherein said determining step further comprises the step of:
calculating the logarithm of the ratio of said second signal to said first signal of each spot.

11. A method for calculating gene expression ratios according to claim 1, further comprising the step of estimating the statistical significance of normalized gene expression ratios.

12. A method for calculating gene expression ratios according to claim 1, wherein said first label is a Cy5 dye.

13. A method for calculating gene expression ratios according to claim 1, wherein said second label is a Cy3 dye.

14. The method of claim 11, wherein said estimating step further comprises:
a) creating at least 10 bins spanning the range of said function of at least one of said first and second signals;
b) calculating the square root of the average variance (s pooled) for each bin created in step a); and
c) estimating the relationship between said function of at least one of said first and second signals and the square root of the average variance using the equation
y=a+b exp(−x/c)
wherein y=square root of the average variance and x is the log of a function of at least one of the signals, and wherein a, b, and c are regression constants.

15. The method according to claim 14, wherein both the s pooled and the regression are weighted to reflect degrees of freedom.

16. The method according to claim 14, wherein said function of at least one of said first and second signals is the logarithm of said first signal.

17. The method according to claim 16, wherein said first signal corresponds to a Cy5 dye.

18. A method for estimating the significance of normalized gene expression ratios from any method for calculating gene expression ratios that comprises:
a) spotting at least one target in at least two or more replicates;
b) calculating the normalized log ratio for each target;
c) calculating one of the s pooled and variance as a function of signal intensity; and
d) determining confidence intervals for gene expression ratios using the value determined in step c).

19. A method for estimating the significance of normalized gene expression ratios from any method for calculating gene expression ratios that comprises:
a) spotting at least one target in at least two or more replicates;
b) calculating the normalized log ratio for each target;
c) calculating one of the s pooled and variance as a function of signal intensity; and
d) flagging unusually variable sets of replicate ratios.
